(19) United States
(12) Patent Application Publication
(10) Pub. No.: US 2008/0159964 A1
(43) Pub. Date: Jul. 3, 2008

(54) USE OF ADENOSINE DEAMINASE FOR TREATING PULMONARY DISEASE

(75) Inventors: Michael R. Blackburn, Pearland, TX (US); Ivan Horak, West Orange, NJ (US); Puja Sapra, Edison, NJ (US)

Correspondence Address: LUCAS & MERCANTI, LLP, 475 PARK AVENUE SOUTH, 15TH FLOOR, NEW YORK, NY, 10016, US

(73) Assignees: Enzon Pharmaceuticals, Inc., Bridgewater, NJ (US); Board of Regents of the University of Texas System, Austin, TX (US)

(21) Appl. No.: 11/965,939

(22) Filed: Dec. 28, 2007

Related U.S. Application Data
(60) Provisional application No. 60/882,748, filed on Dec. 29, 2006.

(51) Int. Cl. A61K 38/45; A61K 9/12; A61P 11/00

(52) U.S. Cl. 424/45; 424/94.5

(57) ABSTRACT

Provided are methods for treating an adenosine deaminase-mediated pulmonary disease such as asthma, pulmonary fibrosis, cystic fibrosis and chronic obstructive pulmonary disease in a mammal in need thereof, by administering and effective amount of a polymer-conjugated adenosine deaminase.

### CROSS-REFERENCE TO RELATED APPLICATION

This application claims the benefit of priority from U.S. Provisional Patent Application Ser. No. 60/882,748 filed Dec. 29, 2006, the contents of which are incorporated herein by reference.

### FIELD OF THE INVENTION

The present invention provides compositions and methods for treating diseases and disorders of the pulmonary system, including, e.g., asthma, pulmonary fibrosis, cystic fibrosis and chronic obstructive pulmonary disease (“COPD”) with adenosine deaminase and/or polymer-conjugated adenosine deaminase.

### BACKGROUND OF THE INVENTION

There are a number of pulmonary diseases and disorders that would benefit from the availability of a selective treatment method that addressed the underlying etiology to treat symptoms with increased effectiveness and reduced side effects of conventional treatments.

Asthma is an inflammatory disease of the airways. In the United States, the disease affects nearly 10 million adults and nearly 5 million children (Redd, 2002, Asthma Occurrence, Environmental Health Perspectives 110 Suppl 4, pp 557-560). The disease is typified by the infiltration and activation of immune cells in the lung, followed by airway inflammation and obstruction (Vogel, 1997, Science 276:1643-1646). Many factors are known to trigger asthma, although the underlying etiology is not well understood. However, Kellems et al., in U.S. Pat. No. 6,207,876, granted on Mar. 27, 2001 (hereinafter “Kellems”), and incorporated by reference herein, provided knockout mice deficient in the adenosine deaminase (“ADA”) enzyme. Data developed from the Kellems ADA deficient mice were reported by that document to confirm a role for adenosine accumulation in the pathophysiology of asthma. Injection of exogenous polymer-conjugated bovine ADA, in the form of polyethylene glycol (“PEG”) conjugated ADA (ADAGEN®, from Enzon Pharmaceuticals, Inc.) was shown by that document to prevent pulmonary accumulation of adenosine, and to reverse inflammatory eosinophilia otherwise present in the ADA deficient mice.

Pulmonary fibrosis is an illness in which the alveoli, or air sacs, of the lungs become inflamed, and are gradually replaced by scar tissue. As the disease progresses, the scar tissue impairs breathing and oxygen transfer. There are a number of known causes, such as cancer, chronic infection or inflammation, industrial dusts, e.g., asbestos, certain drugs, and the like. Current treatments include long term administration of non-specific antiinflammatory/antimitotic agents such as glucocorticosteroids, cyclophosphamide, azathioprine, colchicine, and the like. These treatments do not always work, and have significant side effects when administered chronically.

Cystic fibrosis (“CF”) is described as the most common, fatal genetic disease in the United States. About 30,000 people in the United States have the disease. CF causes the body to produce thick, sticky mucus that clogs the lungs, leads to infection, and blocks the pancreas, which stops digestive enzymes from reaching the intestine where they are required in order to digest food. Previously, there have been no effective methods of treating the symptoms of this disease. Current palliative treatments include diet modifications, and nonspecific measures to loosen and free up the dangerous secretions.

Chronic obstructive pulmonary disease (“COPD”) is stated by the U.S. National Heart, Lung and Blood Institute of the NIH, to be the fourth leading cause of death in the United States and throughout the world. COPD is a lung disease in which both bronchioles as well as terminal bronchioles and their respective alveoli are damaged, so that respiration is impaired. Cigarette smoking is the most common cause of COPD, although chronic exposure to other pulmonary irritants, such as air pollution, dust, or chemicals, over a long period of time, may also cause or contribute to COPD. Previously, there has been no effective treatment for COPD, with patients being managed with palliative bronchodilators, nonsteroidal antiinflammatory agents, and corticosteroid antiinflamatory agents, as well as with supplemental oxygen.

Adenoside deaminase, or ADA, also known as adenosine aminohydrolase is designated as EC 3.5.4.4 (SEQ ID NO: 1 illustrates the peptide sequence of natural bovine ADA). ADA converts either adenosine or deoxyadenosine, in the presence of water, into inosine or deoxyinosine and ammonia, and is therefore important to the purine salvage pathway. Polymer-conjugation of ADA minimizes the possibility of a deleterious antigenic response to an injected bovine protein, as well as improving the kinetics of the enzyme after injection. ADAGEN® is presently approved by the U.S. Food and Drug Administration as an orphan drug in the treatment of severe combined immune deficiency, or SCID (also art-known as “bubble boy syndrome”). SCID has been shown to be caused by a deficiency of endogenous ADA in SCID patients.

Thus, for all of the foregoing reasons, there remains a long sought need for a new treatment for pulmonary diseases, as listed above, as well as for a successful administration of inhaled ADAGEN® for treating such disorders.

### SUMMARY OF THE INVENTION

There are provided methods of treating adenosine demainase-mediated pulmonary diseases such as asthma, pulmonary fibrosis, cystic fibrosis, chronic obstructive pulmonary disease and related conditions in a mammal in need thereof. In alternative aspects, the present invention provides methods of treating pulmonary diseases associated with elevated levels of adenosine. The methods include administering an effective amount of adenosine deaminase to the mammals in need thereof. The methods contemplated herein include administering the adenosine deaminase one or more times daily for one or more days, including daily administrations for extended periods until such time as the disease is abated. In some aspects of this embodiment, the adenosine deaminase is administered by inhalation or injection. In some preferred aspects, the enzyme is administered via inhalation using art recognized devices, i.e. inhalers or the like, for pulmonary delivery of sufficient amounts of the enzyme as an aerosol or as a dry powder. Alternatively, the adenosine deaminase or conjugate thereof is administered parenterally such as via the intravenous route.

In further aspects of the invention, the adenosine deaminase can be obtained from a bovine source, a human source or other suitable mammalian source. Recombinant forms of the adenosine deaminase are also contemplated.

The adenosine deaminase can preferably be conjugated to a polyalkylene oxide, such as polyethylene glycol which can be straight, branched or multi-arm polymers. Suitable polyalkylene oxides and PEG's will have molecular weights ranging from about 2,000 to about 45,000 daltons. In some especially preferred aspects of the invention, the adenosine deaminase conjugated to polyethylene glycol is ADAGEN® (pegademase bovine) available from Enzon Pharmaceuticals, Inc., of Bridgewater, N.J. USA.

As described herein, the amount of adenosine deaminase administered to the mammal, preferably a human, is an amount sufficient to maintain plasma ADA activity (trough levels) in the range of from about 15 to about 35 μmol/hr/mL (assayed at 37° C.); and demonstrate a decline in adenosine such as erythrocyte dATP to ≦ about 0.005—about 0.015 μmol/mL in packed erythrocytes, or ≦ about 1% of the total erythrocyte adenine nucleotide (i.e., ATP+dATP content), with a normal adenosine level, as measured in a pre-injection sample. Stated in an alternative manner, the amount of adenosine deaminase administered to the patient is an amount sufficient to reduce lung adenosine levels to less than about 10 nmoles per mg protein, and more preferably an amount sufficient to reduce lung adenosine levels to less than about 5 nmoles per mg protein.

Alternative embodiments of the invention include administering an effective dose of a second pharmacologically active agent in combination with the adenosine deaminase to the patients in need thereof. Suitable second pharmacologically active agents include bronchodilators such as theophylline or other well known bronchodilating agents having beta-adrenergic properties such as salmeterol, albuterol or terbutaline.

Still further aspects of the invention include kits for treating pulmonary disease in mammals, inhalable formulations comprising adenosine deaminase and a bronchodilator; and inhalers suitable for use in the treatment of pulmonary conditions, comprising the inhalable formulations described herein and a propellant.

For purposes of the present invention, the term “adenosine” shall be understood to mean adenosine and deoxyadenosine. The adenosine also includes adenosine and deoxyadenosine present in the form of AMP, ADP, ATP, dAMP, dADP or dATP.

For purposes of the present invention, the term “residue” shall be understood to mean that portion of a compound, to which it refers, i.e. PEG, oligonucleotide, etc. that remains after it has undergone a substitution reaction with another compound.

For purposes of the present invention, the term “polymeric residue” or “PEG residue” shall each be understood to mean that portion of the polymer or PEG which remains after it has undergone a reaction with other compounds, moieties, etc.

For purposes of the present invention, the term “alkyl” as used herein refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, C₁₋₆ hydrocarbonyl, groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, tribalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” as used herein refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

The term “alkenyl” as used herein refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

The term “alkynyl” as used herein refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarb on rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkyl” as used herein refers to a C₃₋₈ cyclic hydrocarbon. Examples of cycloalkyl include cyclopropyl, cyclobutyl, cyclopentyl, cyclohexyl, cycloheptyl and cyclooctyl.

The term “cycloalkenyl” as used herein refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

The term “cycloalkylalkyl” as used herein refers to an alklyl group substituted with a C₃₋₈ cycloalkyl group. Examples of cycloalkylalkyl groups include cyclopropylmethyl and cyclopentylethyl.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

An “alkylaryl” group as used herein refers to an aryl group substituted with an alkyl group.

An “aralkyl” group as used herein refers to an alkyl group substituted with an aryl group.

The term “alkoxyalkyl” group as used herein refers to an alkyl group substituted with an alkloxy group.

The term “alkyl-thio-alkyl” as used herein refers to an alkyl-S-alkyl thioether, for example methylthiomethyl or methylthioethyl.

The term “amino” as used herein refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

The term “alkylcarbonyl” as used herein refers to a carbonyl group substituted with alkyl group.

The terms “halogen” or “halo” as used herein refer to fluorine, chlorine, bromine, and iodine.

The term “heterocycloalkyl” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “heteroatom” as used herein refers to nitrogen, oxygen, and sulfur.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

For purposes of the present invention, “positive integer” shall be understood to include an integer equal to or greater than 1 and as will be understood by those of ordinary skill to be within the realm of reasonableness by the artisan of ordinary skill.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art.

### BRIEF DESCRIPTION OF THE DRAWINGS

FIG. 1A and FIG. 1B show therapeutic effects of adenosine deaminase polymer conjugates on pulmonary inflammation and fibrosis described in Example 1.

FIG. 2 shows effects of adenosine deaminase polymer conjugates on adenosine levels in mice with pulmonary fibrosis described in Example 2.

FIG. 3 shows effects of adenosine deaminase polymer conjugates on weight loss in mice with pulmonary fibrosis described in Example 3.

FIG. 4A and FIG. 4B show therapeutic effects of adenosine deaminase polymer conjugates on inflammation in mice with pulmonary fibrosis described in Example 4.

FIG. 5 shows effects of adenosine deaminase polymer conjugates on collagen production in mice with pulmonary fibrosis described in Example 5.

### DETAILED DESCRIPTION OF THE INVENTION

Accordingly, the invention provides new methods and compositions for the treatment of pulmonary diseases and disorders including, e.g., asthma, pulmonary fibrosis, cystic fibrosis, and COPD, by administering ADA enzyme to a patient in need thereof, in an amount, and for a duration, sufficient to reduce the amount of adenosine present in the tissues and/or body fluids of the patient. Preferably, the ADA enzyme is polymer-conjugated. In further embodiments, the ADA enzyme is administered by injection or inhalation. For those pulmonary disease processes that depend upon the presence of endogenous adenosine to sustain the underlying pulmonary pathology, a sufficient reduction in endogenous adenosine by means of administered ADA will treat the symptoms and/or signs of the disease.

As used herein, “adenosine deaminase mediated pulmonary disease” shall be understood as broadly including any pulmonary disease, condition or disorder which benefits from the administration of ADA, or active fraction thereof, etc., regardless of the route of administration. Such pulmonary diseases are not limited to those which are strictly associated with increased levels of adenosine in the lungs, bronchioles, alveoli or related tissues.

The administration of the ADA enzyme according to the methods of the invention may be for either a “prophylactic” or “therapeutic” purpose. When provided prophylactically, the ADA enzyme is provided in advance of any pulmonary symptom. The prophylactic administration of the agent(s) serves to prevent or attenuate any subsequent pulmonary symptom(s). When provided therapeutically, the ADA enzyme is provided at (or shortly after) the onset of a symptom of asthma. The therapeutic administration of the ADA enzyme serves to attenuate any actual pulmonary symptom episode. The methods of the present invention may, thus, be carried out either prior to the onset of an anticipated pulmonary symptom (so as to attenuate the anticipated severity, duration or extent of the symptom) or after the initiation of the symptom.

In yet alternative aspects, the ADA conjugates according to the methods described herein can be used in combination, simultaneously or sequentially, with a chemotherapeutic agent treatment. Serious complications can occur in the lungs by chemotherapeutic agents. For example, bleomycin marketed under the brandname, BLENOXANE is known to cause pulmonary fibrosis and impair lung function. The ADA conjugates described herein can attenuate, reduce or prevent pulmonary disease associated with chemotherapy. Thus, the ADA enzyme according to the methods described herein can be administered prophylactically, concurrently or after the administration of the chemotherapeutic agent.

Successful treatment of pulmonary disease shall be deemed to occur when at least 20% or preferably 30%, more preferably 40% or higher (i.e., 50% or 80%) decrease in adenosine, inflammatory cells, and/or fibrosis including other clinical markers contemplated by the artisan in the field is realized when compared to that observed in the absence of the ADA treatment. Other endpoints include the degree of extracellular matrix production deposition, fibroblast numbers, proteinase antiproteinase enzyme levels, levels of profibrotic mediators, and histopathological evidence of pulmonary obstruction. Airway remodeling and/or destruction are also tractable endpoints.

### A. ADA Polymer Conjugates

Broadly speaking, methods and compositions for reducing systemic or local adenosine levels are provided for treating diseases or disorders of the pulmonary system.

In one aspect, pharmaceutical compositions for use according to the invention include an ADA polypeptide, or an active fragment thereof including variations, polymorphisrms and derivatives thereof. Preferably, the ADA is bovine or human ADA although other mammalian species are contemplated. In one preferred embodiment, the ADA is purified from bovine sources. The Cys 74 residue of the naturally occurring bovine ADA is capped or protected by a cysteine and the six C-terminal residues predicted from the gene encoding the ADA of SEQ ID NO: 1 are not present. In those aspects where animal source ADA is used, it is obtained, purified, etc., i.e. from cows, etc., using techniques known to those of ordinary skill. In a further aspect, it is contemplated that the invention can employ alternative variations on natural bovine ADA including alternative alleles and polymorphisms with and without the predicted six C-terminal residues. Bovine ADA polymorphisms include, e.g., glutamine at position 198 in place of lysine, alanine at position 245 in place of threonine, arginine at position of 351 instead of glycine.

In alternative aspects, the ADA is a recombinant ADA. For example, the adenosine deaminase can be a recombinant bovine adenosine deaminase (SEQ ID NO: 1) or a recombinant human adenosine deaminase (“rhADA”, SEQ ID NO: 3) translated from a DNA molecule according to SEQ ID NO: 2 or SEQ ID NO: 4. Optionally, the recombinant adenosine deaminase can lack the six C-terminal residues of the bovine adenosine deaminase.

In a further aspect of the invention, derivatives of ADA enzyme can include recombinantly produced ADA enzyme that has been mutated for enhanced stability relative to nonmutated recombinant ADA enzyme. These include, for example, recombinant ADA enzymes modified from SEQ ID NO: 1 and/or SEQ ID NO: 1 with one or more of the above-noted polymorphisms, to replace an oxidizable Cys residue that is solvent-exposed with a suitable non-oxidizable amino acid residue. Such non-oxidizable amino acid residue includes any art-known natural amino acid residue and/or any art-known derivatives thereof. Preferred naturally-occurring amino acids optionally substituted for cysteine in recombinant ADA, include, e.g., alanine, serine, asparagine, glutamine, glycine, isoleucine, leucine, phenylalanine, threonine, tyrosine, and valine. Serine is most preferred. Some preferred recombinant ADA mutein enzymes are illustrated by SEQ ID NO: 5 (bovine ADA) and SEQ ID NO: 7 (human ADA) translated from a DNA molecule according to SEQ ID NO: 6 or SEQ ID NO: 8. Additional details concerning such recombinant ADA muteins, and production and purification of these proteins, are provided by co-owned U.S. Application Nos. 60/913,009 and 60/913,039, incorporated by reference herein in their entirety. Specific details on the vectors and method of purification are found therein, particularly in the Examples section, and most particularly in Examples 1-4 of the '009 application.

In a further aspect, the recombinant ADA can be stabilized by capping a solvent-exposed oxidizable Cys reside. An oxidizable amino acid such as cysteine residue of the recombinant ADA can be capped by the capping agent such as oxidized glutathione, iodoacetamide, iodoacetic acid, cystine, other dithiols and mixtures thereof without substantially inactivating the ADA protein. The capping of the recombinant ADA stabilizes and protects the ADA from degradation. Details of capping the ADA are described in U.S. patent application Ser. No. 11/738,012, the contents of which are incorporated herein by reference.

In preferred aspects, the ADA polypeptide is conjugated to a substantially non-antigenic polymer, preferably a polyalkylene oxide (“PAO”).

The ADA-polymer conjugates generally correspond to formula (I):

[R—NH]_{z}-(ADA) (I)

wherein

(ADA) represents the adenosine deaminase or active fragment thereof, either a purified form from such as bovine or a recombinant ADA;

NH— is an amino group of an amino acid found on the ADA for attachment to the polymer;

z is a positive integer, preferably from about 1 to about 80; and

R includes a substantially non-antigenic polymer residue that is attached to the ADA in a releasable or non-releasable form.

In more preferred aspects, the polymers include polyethylene glycol (PEG) wherein the PEG can be linear, branched or multi-armed PEG. Generally, polyethylene glycol has the formula:

—O—(CH₂CH₂O)ₙ—

wherein (n) is a positive integer, preferably from about 10 to about 2,300. The average molecular weight of the polymers ranges from about 1000 to about 100,000 Da. More preferably, the polymers have an average molecular weight of from about 5,000 Da to about 45,000 Da, yet more preferably, 5,000 Da to about 20,000 Da. Most preferably, the PEG is about 5,000 Daltons, as is found in ADAGEN® (pegylated bovine adenosine deaminase). Other molecular weights are also contemplated so as to accommodate the needs of the artisan.

Alternatively, the polyethylene glycol (PEG) residue portion of the invention can be represented by the structure:

—Y₇₁—(CH₂CH₂O)ₙ—CH₂CH₂Y₇₁—,

—Y₇₁—(CH₂CH₂O)ₙ—CH₂C(═Y₇₂)—Y₇ₗ—,

—Y₇₁—C(═Y₇₂)—(CH₂)ₐ₇₁—Y₇₃—(CH₂CH₂O)ₙ—CH₂CH₂—Y₇₃—(CH₂)ₐ₇₁—C(═Y₇₂)—Y₇₁—,

—Y₇₁—(CR₇₁R₇₂)ₐ₇₂—Y₇₃—(CH₂)_{b71}—O—(CH₂CH₂O)ₙ—(CH₂)_{b71}—Y₇₃—(CR₇₁R₇₂)ₐ₇₂—Y₇₁—,

—Y₇₁—(CH₂CH₂O)ₙ—CH₂CH₂—,

—Y₇₁—(CH₂CH₂O)ₙ—CH₂C(═Y₇₂)—,

—C(═Y₇₂)—(CH₂)ₐ₇₁—Y₇₃—(CH₂CH₂O)ₙ—CH₂CH₂—Y₇₃—(CH₂)ₐ₇₁—C(═Y₇₂)—, and

—(CR₇₁R₇₂)ₐ₇₂—Y₇₃—(CH₂)_{b71}—O—(CH₂CH₂O)ₙ—(CH₂)_{b71}—Y₇₃—(CR₇₁R₇₂)ₐ₇₂—,

wherein:

Y₇₁ and Y₇₃ are independently O, S, SO, SO₂, NR₇₃ or a bond;

Y₇₂ is O, S, or NR₇₄;

R₇₁₋₇₄ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;

(a71), (a72), and (b71) are independently zero or a positive integer, preferably 0-6, and more preferably 1; and

(n) is an integer from about 10 to about 2300.

As an example, the PEG can be functionalized in the following non-limiting manner:

—C(═Y₇₄)—(CH₂)ₘ—(CH₂CH₂O)ₙ—,

—C(═Y₇₄)—Y—(CH₂)ₘ—(CH₂CH₂O)ₙ—,

—C(═Y₇₄)—NR₁₁—(CH₂)ₘ—(CH₂CH₂O)ₙ—,

—CR₇₅R₇₆—(CH₂)ₘ—(CH₂CH₂O)ₙ—

wherein

R₇₅ and R₇₆ are independently selected from among H, C₁₋₆ alkyls, aryls, substituted aryls, aralkyls, heteroalkyls, substituted heteroalkyls and substituted C₁₋₆ alkyls;

m is zero or is a positive integer, and preferably 1;

Y₇₄ is O or S; and

n represents the degree of polymerization.

In a further aspect, the polymer portion of the conjugate can be one which affords multiple points of attachment for the ADA. Alternatively, multiple PEGs can be attached to the ADA.

The pharmacokinetics and other properties of PEGylated ADA can be adjusted as needed for a desired clinical application by manipulation of the PEG molecular weight, linker chemistry and ratio of PEG chains to enzyme.

In these aspects, the ADA can be attached to the non-antigenic polymer in releasable or non-releasable form via various linkers known in the art.

The releasable polymer systems can be based on benzyl elimination or trimethyl lock lactonization. The activated polymer linkers of the releasable polymer systems can be prepared in accordance with commonly-assigned U.S. Pat. Nos. 6,180,095, 6,720,306, 5,965,119, 6,624,142 and 6,303,569, the contents of which are incorporated herein by reference. Alternatively, the ADA polymer conjugates are made using certain bicine polymer residues such as those described in commonly assigned U.S. Pat. Nos. 7,122,189 and 7,087,229 and U.S. patent application Ser. Nos. 10/557,522, 11/502,108, and 11/011,818, incorporated by reference herein. Other releasable polymer systems contemplated are also described in PCT/US07/78600, the contents of which are incorporated herein by reference.

Illustrative examples of releasable or non-releasable ADA polymer conjugates contemplated herein are described in U.S. Patent Application No. 60/913,039, the contents of which are incorporated herein by reference.

For purposes of the present invention, those polymers should be functionalized or activated to attach the ADA. Those of ordinary skill can use various activated forms of the polymers for attachment without undue experimentation. Some preferred activated PEGs include those disclosed in commonly assigned U.S. Pat. Nos. 5,122,614, 5,324,844, 5,612,460 and 5,808,096, the contents of which are incorporated herein by reference. For example, Zalipsky, in U.S. Pat. No. 5,122,614, describes the activation of PEG by conversion into its N-succinimide carbonate derivative (“SC-PEG”).

As will be appreciated by those of ordinary skill such conjugation reactions typically are carried out in a suitable buffer using a several-fold molar excess of activated PEG. Some preferred conjugates made with linear PEGs like the above mentioned SC-PEG can contain, on average, from about 20 to about 80 PEG strands per enzyme. Consequently, for these, molar excesses of several hundred fold, e.g., 200-1000× can be employed. The molar excess used for branched PEG and PEG attached to the enzyme will be lower and can be determined using the techniques described in the patents and patent applications describing the same that are mentioned herein.

In these aspects, the polyalkylene oxide is conjugated to the protein via linker chemistry including, e.g., succinimidyl carbonate, thiazolidine thione, urethane, and amide based linkers. The polyalkylene oxide is preferably covalently attached to an epsilon amino group of a Lys on the ADA purified from bovine or the cysteine-stabilized recombinant human adenosine deaminase, although other sites for covalent attachment are well known to the art. The capped ADA polymer conjugates can include at least 5 polyethylene glycol strands attached to epsilon amino groups of Lys on the enzyme, but alternatively, can include about 11-18 PEG strands attached to epsilon amino groups of Lys on the enzyme.

While the ADA of ADAGEN® is conjugated to from about 11 to about 18 PEG molecules per enzyme molecule, via lysine linkages, the ratio of PEG to ADA can be varied in order to modify the physical and kinetic properties of the combined conjugate to fit any particular clinical situation.

It will be apparent from the foregoing that additional aspects of the invention include using any commercially available or reported activated PEG or similar polymer to conjugate the ADA enzyme or fragment thereof in order to provide conjugates useful for the methods of treatment described herein. See, e.g., the Nektar Advanced Pegylation catalog of 2004 (Nektar, San Carlos, Calif.), incorporated by reference herein in its entirety.

In another aspect, the activated polymer linkers are prepared using branched polymer residues such as those described commonly assigned U.S. Pat. Nos. 5,643,575, 5,919,455, 6,113,906 and 6,566,506, the disclosure of each being incorporated herein by reference. A non-limiting list of such polymers corresponds to polymer systems (i)-(vii) with the following structures:

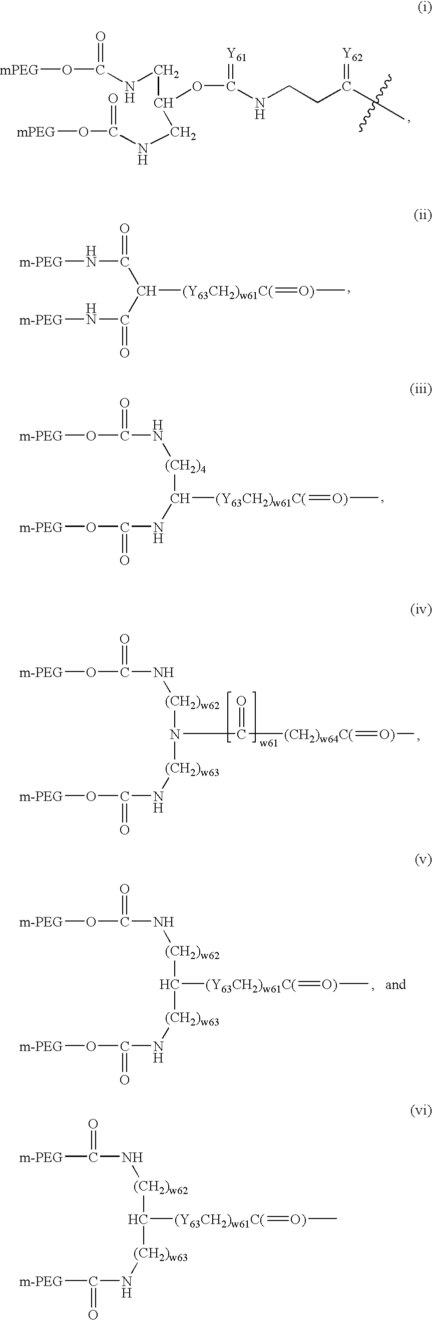

wherein:

Y₆₁₋₆₂ are independently O, S or NR₆₁;

Y₆₃ is O, NR₆₂, S, SO or SO₂

(w62), (w63) and (w64) are independently 0 or a positive integer, preferably from about 0 to about 10, more preferably from about 1 to about 6;

(w61) is 0 or 1;

mPEG is methoxy PEG
wherein PEG is previously defined and a total molecular weight of the polymer portion is from about 1,000 to about 100,000 daltons; and

R₆₁ and R₆₂ are independently the same moieties which can be used for R₇₁.

Also useful are multi-arm PEG derivatives such as “star-PEG's” and multi-armed PEG's described in Shearwater Corporation's 2001 catalog “Polyethylene Glycol and Derivatives for Biomedical Application”. See also NOF Corp. Drug Delivery System catalog, Ver. 8, April 2006. The disclosure of each of the foregoing is incorporated herein by reference. The multi-arm polymers contain four or more-polymer arms and preferably four or eight polymer arms.

For purposes of illustration and not limitation, the multi-arm polyethylene glycol (PEG) residue can be

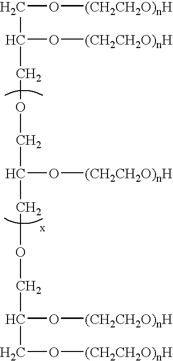

wherein:

(x) is 0 and a positive integer, i.e. from about 0 to about 28; and

(n) is the degree of polymerization.

In one particular embodiment of the present invention, the multi-arm PEG has the structure:

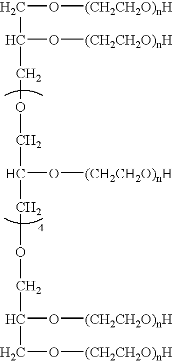

wherein n is a positive integer. In one preferred embodiment of the invention, the polymers have a total molecular weight of from about 1,000 Da to about 100,000 Da, and preferably from 5,000 Da to 45,000 Da.

In another particular embodiment, the multi-arm PEG has the structure:

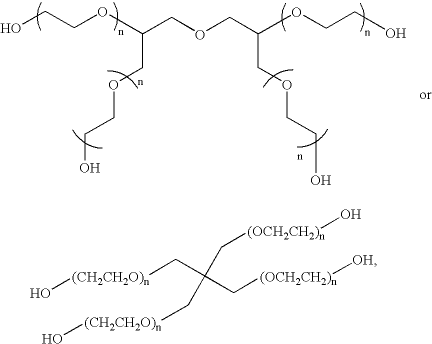

wherein n is a positive integer.

The polymers can be converted into a suitably activated polymer, using the activation techniques described in U.S. Pat. Nos. 5,122,614 or 5,808,096. Specifically, such PEG can be of the formula:

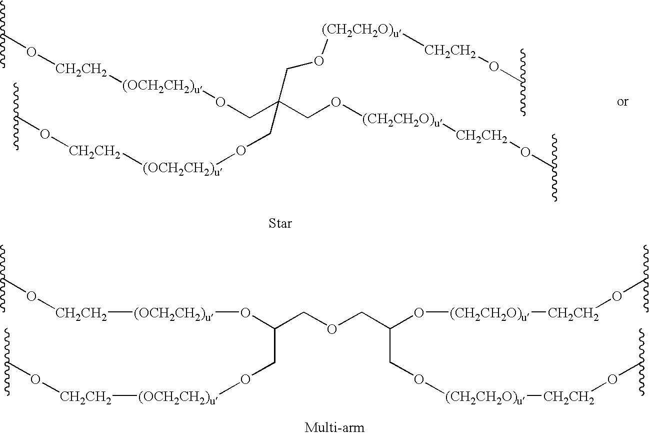

wherein:

(u′) is a positive integer; and up to 3 terminal portions of the residue is/are capped with a methyl or other lower alkyl.

In some preferred embodiments, all four of the PEG arms can be converted to suitable activating groups, for facilitating attachment to ADA. Such compounds prior to conversion include:

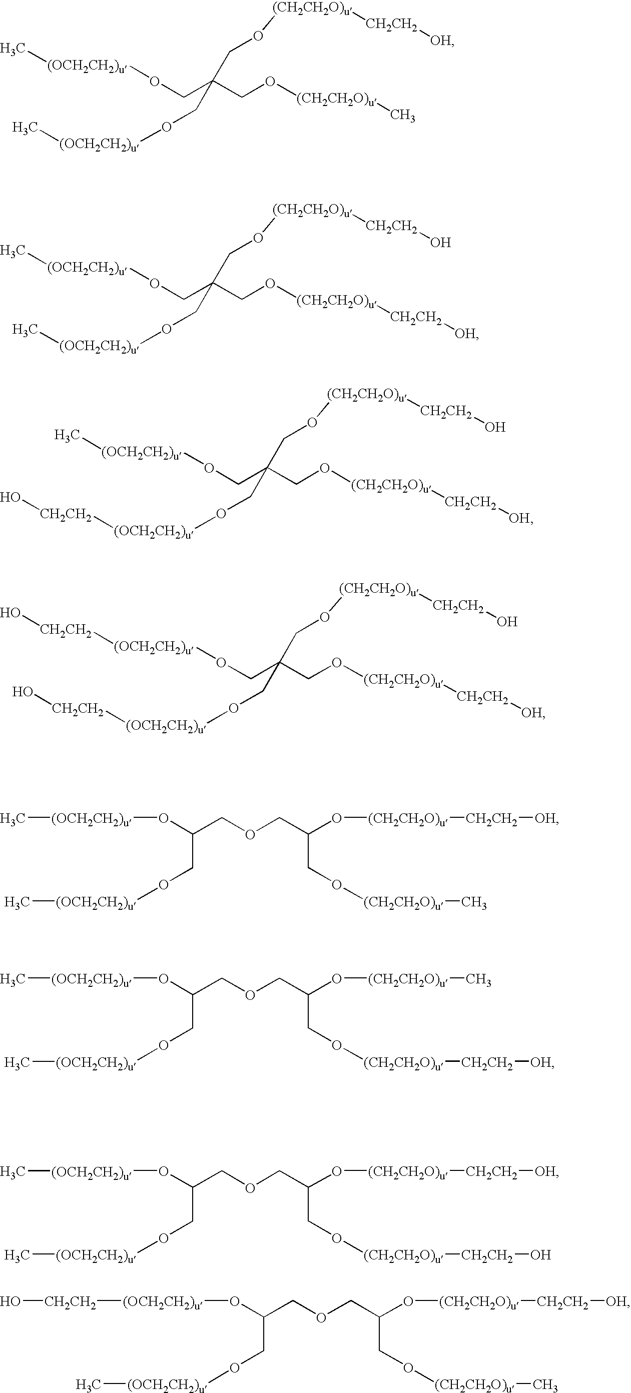

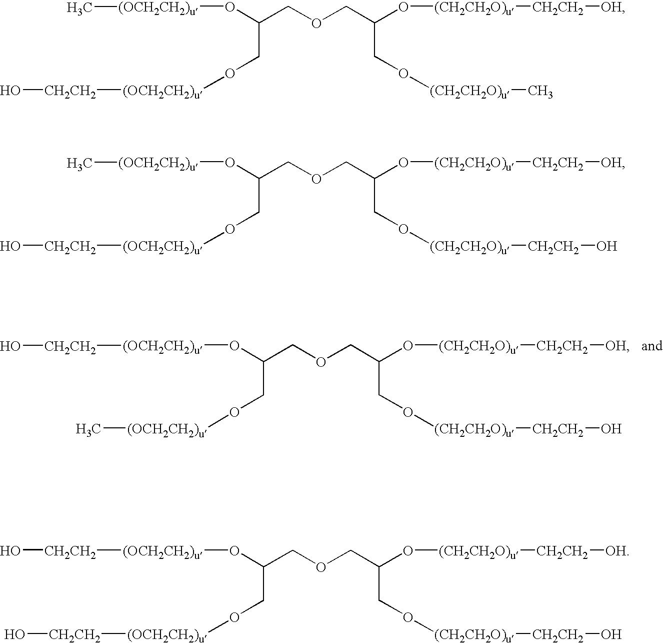

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In a further embodiment, and as an alternative to PAO-based polymers, other suitable polymers are each optionally selected from among one or more effectively non-antigenic materials such as dextran, polyvinyl alcohols, carbohydrate-based polymers, hydroxypropylmeth-acrylamide (HPMA), polyalkylene oxides, and/or copolymers thereof.

See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated and that other polyalkylene oxide derivatives such as the polypropylene glycols; etc. are also contemplated.

### B. Pharmaceutical Compositions

The ADA or ADA polymer-conjugate pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy delivery by syringe exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In a preferred embodiment, the ADA polypeptide is conjugated to PEG. Under ordinary conditions of storage and use, these preparations contain a preservative to prevent the growth of microorganisms.

An ADA enzyme as described supra be formulated into a composition in a neutral or salt form. Pharmaceutically acceptable salts, include the acid addition salts (formed with the free amino groups of the protein). Some suitable inorganic acids include for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The use of peptide therapeutics as active ingredients is described in greater detail by the technology of U.S. Pat. Nos. 4,608,251; 4,601,903; 4,599,231; 4,599,230; 4,596,792; and 4,578,770, each incorporated herein by reference.

The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The ADA PEG-conjugate or other ADA-containing therapy may be formulated within a therapeutic mixture to comprise from about 100 U to about 300 U per ml, and preferably at about 250 U/ml, wherein one unit of activity is defined as the amount of ADA that converts 1 μM of adenosine to inosine per minute at 25° C. and pH 7.3 as indicated for intercurrent illnesses.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used.

In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

In providing the ADA or ADA PEG-conjugate by injection, it is generally desirable to provide the recipient with a dosage that will 1) maintain plasma ADA activity in the range of from about 10 to 100 μmol/hr/mL, preferably from about 15 to about 35 μmol/hr/mL (assayed at 37° C.); and 2) demonstrate a decline in erythrocyte adenosine, i.e., dATP to ≦ about 0.001-0.057 μmol/mL, preferably about 0.005—about 0.015 μmol/mL in packed erythrocytes, or ≦ about 1%, of the total erythrocyte adenosine (i.e., ATP+dATP content), with a normal adenosine level, as measured in a pre-injection sample. The normal value of dATP is below about 0.001 μmol/mL.

The methods contemplated herein can include administering the adenosine deaminase one or more times (i.e. twice) weekly for one or more weeks until such time as the pulmonary disease is abated. The compositions may be administered once daily or divided into multiple doses which can be given as part of a multi-week treatment protocol.

### C. Delivery by Inhalation

Pulmonary drug delivery can be achieved by different approaches, including liquid nebulizers, aerosol-based metered dose inhalers (MDI's) using air or other propellant, e.g., HFA-134a (1,1,1,2-tetrafluoroetliane). Dry powder dispersion devices are also available. Dry powder dispersion devices, are employed to deliver drugs that are readily formulated as dry powders, particularly proteins and polypeptides. Many otherwise labile proteins and polypeptides may be stably stored as lyophilized or spray-dried powders by themselves or in combination with suitable powder carriers.

In providing a patient with inhaled ADA or PEG-conjugated ADA enzyme capable of reducing pulmonary adenosine levels, the dosage of administered agent will vary depending upon such factors as the patient's age, weight, height, sex, general medical condition, previous medical history, etc. The artisan will appreciate the need to titrate the initial dose to desired clinical endpoints to achieve and maintain reduction of symptoms, by administering inhaled ADA or ADA PEG-conjugate, e.g., ADAGEN® at a dose and for a time period effective to achieve such clinical endpoints, while avoiding or n minimizing any side effect that may develop.

When administered as a dry powder, e.g., by a metered dose system, the dose, based on the amount of enzyme, will range from, for example, about 0.10 U/kg through about 30 U/kg, preferably from about 0.5 U/kg through about 20 U/kg, more preferably from about 0.5 U/kg through about 10 U/kg (i.e. per kg of patient body weight), and yet more preferably from about 0.5 U/kg through about 5 U/kg. ADA dosage information is also described in the prescription insert for ADAGEN®, the contents of which are incorporated herein.

When administered from a nebulized solution, the does will range from, for example, about 0.01 U/kg through about 5 U/kg. More preferably, from about 0.1 U/kg through about 1 U/kg.

In certain embodiments, the inhaled ADA or PEG-conjugated ADA enzyme can be administered in combination or alongside therapy with other art known pulmonary agents, administered orally, by injection, e.g., subcutaneously, intravenously and/or intramuscularly, and/or by inhalation. Such agents include bronchodilators glucocorticoids, and the like, as described in greater detail by Goodman and Gilman's, the Pharmacological Basis of Therapeutics, Eleventh Edition, Publ. McGraw Hill, incorporated by reference herein in its entirety. In particular, such additional agents include, by way of example, methylxanthines (such as theophylline), beta-adrenergic agonists (such as catecholamines, resorcinols, saligenins, and ephedrine), selective beta adrenergic agonist, such as albuterol, terbutaline, and the like, glucocorticoids (such as hydrocortisone), other inhalable steroids used for treatment of pulmonary conditions, chromones (such as cromolyn sodium) and anticholinergics (such as atropine), or any other pulmonary agent, in order to decrease the amount of such agents needed to treat the symptoms of a pulmonary disease or disorder. As used herein, one compound is said to be additionally administered with a second compound when the administration of the two compounds is in such proximity of time that both compounds can be detected at the same time in the patient's serum. Pre-administration of a bronchodilator, such as the above-noted methylxanthines, beta-adrenergic agonists, and the like, is optionally preferred to aid in the penetration of inhaled ADA PEG conjugate to sites of action within the bronchioles and alveoli. A non-limiting list of suitable secondary pharmacologically active agents which can be employed herein, therefore include aminophylline, theophylline, bitolerol, dyphylline, formoterol, ipratropium, levalbuterol, metoproterenol, pirbuterol, salmeterol, terbutaline, as well as all other agents known to those of ordinary skill to be useful in the treatment of the pulmonary conditions described herein.

### 1. Dry Powder Delivery of ADA or PEG-Conjugated ADA Enzyme for Inhalation

ADA or ADA PEG-conjugates for inhalation are prepared for dry dispersal, for example, by spray drying a solution containing ADA or ADA PEG-conjugate using methods according to U.S. Pat. Nos. 6,509,006, 6,592,904, 7,097,827 and 6,358,530, all incorporated by reference herein. These patents provide methods and excipients that aid in the dispersal of protein therapeutics for administration by inhalation. Exemplary dry powder excipients include a low molecular weight carbohydrate or polypeptide to be mixed with the ADA or ADA PEG-conjugate to aid in dispersal.

The types of pharmaceutical excipients that are useful as carriers for dry powder dispersal include stabilizers such as human serum albumin (HSA), that is also a useful dispersing agent, bulking agents such as carbohydrates, amino acids and polypeptides; pH adjusters or buffers; salts such as sodium chloride; and the like. These carriers may be in a crystalline or amorphous form or may be a mixture of the two.

Bulking agents which may be combined with the powders include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-beta-cyclodextrin; and polysaccharides, such as raffinose, maltodextrins, dextrans, and the like; alditols, such as mannitol, xylitol, and the like. A preferred group of carbohydrates includes lactose, trehalose, raffinose maltodextrins, and mannitol. Suitable polypeptides include aspartame. Amino acids include alanine and glycine, with glycine being preferred.

Additives may be included for conformational stability during spray drying and for improving dispersibility of the powder, e.g., hydrophobic amino acids such as tryptophan, tyrosine, leucine, phenylalanine, and the like. Suitable pH adjusters or buffers include organic salts prepared from organic acids and bases, such as sodium citrate, sodium ascorbate, and the like; sodium citrate is preferred.

Unit dosage forms for pulmonary delivery comprise a unit dosage receptacle containing a dry powder as described above. The powder is placed within a suitable dosage receptacle in an amount sufficient to provide a subject with drug for a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. Nos. 4,227,522, 4,192,309, and 4,105,027, incorporated by reference herein in their entireties. Suitable containers also include those used in conjunction with Glaxo's Ventolin Rotohaler brand powder inhaler or Fison's Spinhaler brand powder inhaler. Another suitable unit-dose container which provides a superior moisture barrier is formed from an aluminum foil plastic laminate. The ADA or ADA PEG-conjugate powder is filled by weight or by volume into the depression in the formable foil and hermetically sealed with a covering foil-plastic laminate. Such a container for use with a powder inhalation device is described in U.S. Pat. No. 4,778,054, incorporated by reference herein, and is used with Glaxo's Diskhaler. See U.S. Pat. Nos. 4,627,432; 4,811,731; and 5,035,237, all incorporated by reference herein. Preferred dry powder inhalers are those described in U.S. patent application Ser. Nos. 08/309,691 and 08/487,184, both incorporated by reference herein. The latter application has been published as WO 96/09085.

The atomization process may utilize any one of several conventional forms of atomizers. Particularly preferred is the use of two-fluid atomization nozzles as described in more detail below which is capable of producing droplets having a median diameter less than 10 microns. The atomization gas will usually be air which has been filtered or otherwise cleaned to remove particulates and other contaminants. Alternatively, other gases, such as nitrogen may be used. The atomization gas will be pressurized for delivery through the atomization nozzle, typically to a pressure above 25 psig, preferably being above 50 psig. Although flow of the atomization gas is generally limited to sonic velocity, the higher delivery pressures result in an increased atomization gas density. Such increased gas density has been found to reduce the droplet size formed in the atomization operation. Smaller droplet sizes, in turn, result in smaller particle sizes. The atomization conditions, including atomization gas flow rate, atomization gas pressure, liquid flow rate, and the like, will be controlled to produce liquid droplets having an average diameter below 11 microns as measured by phase doppler velocimetry. In defining the preferred atomizer design and operating conditions, the droplet size distribution of the liquid spray is measured directly using Aerometric's Phase Doppler Particle Size Analyzer. The droplet size distribution may also be calculated from the measured dry particle size distribution (Horiba Capa 700) and particle density. The results of these two methods are in good agreement with one another. Preferably, the atomized droplets will have an average diameter in the range from 5 microns to 11 microns, more preferably from 6 microns to 8 microns. The gas:liquid mass flow ratio is preferably maintained above 5, more preferably being in the range from 8 to 10. Control of the gas:liquid mass flow ratio within these ranges is particularly important for control of the particle droplet size.

The liquid medium may be a solution, suspension, or other dispersion of the ADA or ADA PEG-conjugate in a suitable liquid carrier. Preferably, the ADA or ADA PEG-conjugate will be present as a solution in the liquid solvent in combination with the pharmaceutically acceptable carrier, and the liquid carrier will be water. It is possible, however, to employ other liquid solvents, such as organic liquids, ethanol, and the like. The total dissolved solids (including the macromolecule and other carriers, excipients, etc., that may be present in the final dried particle) may be present at a wide range of concentrations, typically being present at from 0.1% by weight to 10% by weight. Usually, however, it will be desirable to maximize the solids concentration that produces particles in the inhalation size range and has the desired dispersibility characteristics, typically the solids concentration ranges from 0.5% to 10%, preferably from 1.0% to 5%. Liquid media containing relatively low concentrations of the ADA or ADA PEG-conjugate will result in dried particulates having relatively small diameters.

Devices for use with the above methods are described, e.g., by U.S. Pat. No. 7,097,827, noted supra.

2. Aerosol Delivery of ADA or PEG-Conjugated ADA Enzyme from Solution or Suspension

In treating pulmonary symptoms, ADA or PEG-conjugated ADA enzyme is preferably administered via an inhaler or nebulizer, in a pharmaceutical formulation suitable for delivery of aerosols in a size range of about 1 micron to about 5 microns in an amount sufficient to lessen or attenuate the severity, extent or duration of the asthma symptoms, employing the dosing guidelines provided supra. In some preferred aspects, the particle size of the ADA-containing formulations PEGylated or not is about 2.5 μm.

Hardware and formulations for the delivery of agents by aerosolized inhalation include, e.g., an aerosol formulation contained in an aerosol container equipped with a metering valve, as described, for example, by U.S. Pat. No. 5,605,674, incorporated by reference herein.

Regardless of the ADA formulation employed, it will be understood that one of the keys associated with inhalation of the enzyme is that a therapeutically effective amount is delivered to and comes in contact with local pulmonary tissue for a period which is sufficient to allow a desirable therapeutic activity to occur. While not wishing to be bound by theory, it is believed that direct exposure of the pulmonary tissue to the ADA results in at least some significant reduction in inflammatory and histopathological conditions in mammals requiring such treatment. Moreover, the invention described herein provides a basis for using ADA in forms such as ADAGEN® in the treatment of certain chronic lung diseases where fibrosis is a major component.

### EXAMPLES

### Example 1

### Efficacy of Systemic Exposure of ADAGEN® in Mice with Pulmonary Inflammation and Fibrosis Caused By Bleomycin

In this example, the efficacy of systemic treatment with ADAGEN® was determined in a mice model with a pulmonary disease such as pulmonary fibrosis. The mice model with pulmonary fibrosis was established by bleomysin. Bleomycin was known to result in pronounced adenosine accumulation and pulmonary fibrosis.

Mice were exposed to saline or bleomycin (dose +2.0 units) intratracheally on day 0. The mice were then treated with systemic ADAGEN® via intraperitoneal injection according to two different treatment regimens; one where treatment was started 3 days following bleomycin exposure (early treatment) to determine if ADAGEN® prevented fibrosis, and a second where treatment was started on day 8 (late treatment) to examine the effects on halting and reversing active disease. For the early treatment, mice were injected with 5 units of ADAGEN® on day 3. For mice with the late treatment, 5 units of ADAGEN® were administered on day 8, 11 and 14. Control mice exposed to saline solution were also examined with and without ADAGEN® treatment.

All analysis was conducted on day 14. Total bronchial alveolar lavage (BAL) cellularity and histopathological differences were examined. The lung cellularity was determined by washing inflammatory cells out of the airway and counting cells using a hemocytometer. Data are presented as mean cell counts+SEM, n=11 for each group. The experiment was repeated twice. For pulmonary histology, lungs were sectioned and stained with heamotoxylin and eosin to examine histopathological changes.

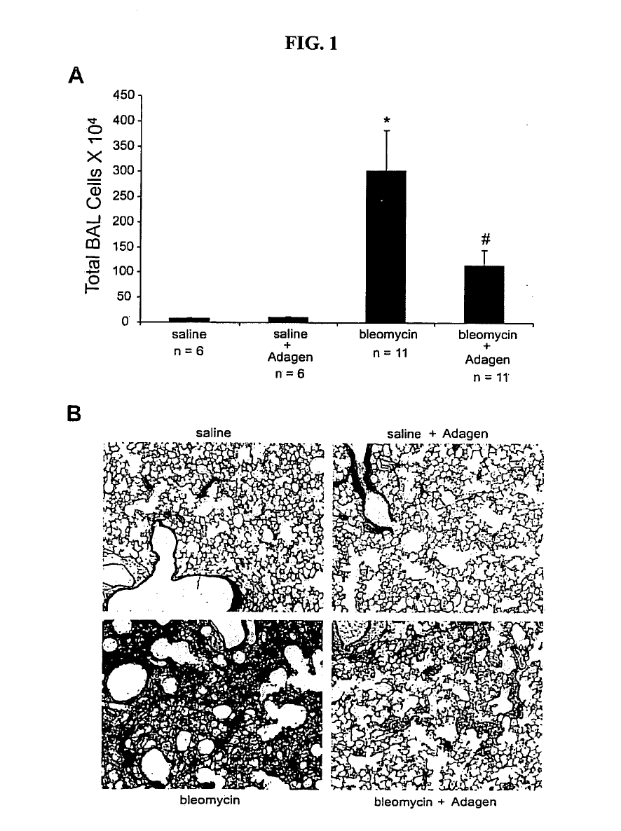

In the mice with pulmonary fibrosis caused by bleomycin, the systemic treatment with ADAGEN® beginning on day 8 resulted in significant reduction in pulmonary inflammation and fibrosis. The results of the late treatment are set forth in FIG. 1A and FIG. 1B.

The results show that the bleomycin exposure increased inflammatory cells in bronchial alveolar lavage. The systemic treatment with ADAGEN® significantly reduced BAL cellularity as shown in (FIG. 1A). Histological analysis of lung tissue revealed severe interstitial inflammation and fibrotic tissue damage in mice exposed to bleomycin. The degree of tissue inflammation and fibrotic damage appeared much less severe in mice treated from day 8 with ADAGEN® (FIG. 1B).

These findings suggest that systemic treatment with ADAGEN® can halt the progression of pulmonary inflammation and fibrosis, and reverse the condition when administered during the fibrotic phase of the disease. These findings show that ADA and ADA polymeric conjugates such as ADAGEN® have utility in the treatment of patients with established pulmonary fibrosis.

### Example 2

### Effects of ADAGEN® Treatment on Adenosine Levels in Mice with Pulmonary Inflammation and Fibrosis Caused by Bleomycin

Adenosine levels were quantified to determine if ADAGEN® treatment lowered adenosine levels in mice model with pulmonary fibrosis caused by bleomycin.

Six week old female C57Blk6 mice were administered 2.0 units of bleomycin intratracheally on day 0. The mice were treated intraperitoneally with an injection of 5 units of ADAGEN® on day 10, 14 and 18 following the bleomycin exposure. Alternatively, mice were treated intraperitoneally with 5 units of ADAGEN® on day 10, 14 and 21 of the protocol. All analysis was conducted on day 21.

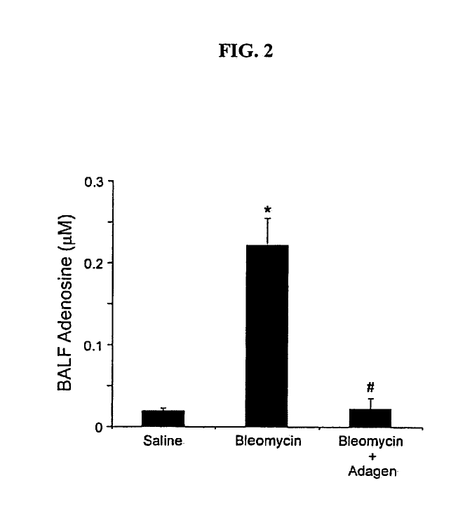

Bronchial alveolar lavage fluid (BALF) was collected from the mice on day 21 and adenosine levels were quantified using reversed phase HPLC. The results are set forth in FIG. 2. Data are presented as mean micromollar concentrations of adenosine+SEM, n=6 for each group. The experiment was repeated twice.

In the mice treated with ADAGEN®, the adenosine was reduced by greater than 90% compared to that of the mice without ADAGEN® treatment. The results demonstrate that ADAGEN® is effective in lowering adenosine levels in mice exposed to bleomycin that exhibit severe pulmonary inflammation and fibrosis.

### Example 3

### Effects of ADAGEN® Treatment on Weight Loss in Mice with Pulmonary Fibrosis Caused by Bleomycin

As an assessment of effects of ADAGEN treatment on the general health of mice with pulmonary fibrosis, body weights were monitored.

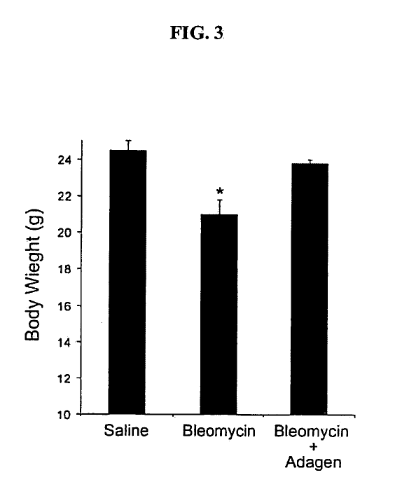

As described in Example 2, the mice exposed to bleomycin were treated intraperitoneally with an injection of 5 units of ADAGEN® on day 10, 14 and 18 following the bleomycin exposure. Alternatively, the mice were treated intraperitoneally with 5 units of ADAGEN® on day 10, 14 and 21. Body weight was measured on day 21 following the bleomycin exposure. The results are set forth in FIG. 3. Data are presented as mean body weights in grams (g)+SEM, n=8 for each group.

There was significant weight loss in mice treated with bleomycin. The weight loss with pulmonary fibrosis caused by bleomycin was prevented by the ADAGEN® treatment, suggesting ADAGEN® treatment was associated with treatment of the disease and improved health.

### Example 4

### Effects of Extended ADAGEN® Treatment on Inflammation in Mice with Pulmonary Fibrosis Caused by Bleomycin

Inflammatory cells were counted to determine if extended ADAGEN® treatment improves lung inflammation in the mice with pulmonary fibrosis.

As described in Example 2, the mice exposed to bleomycin were treated intraperitoneally with an injection of 5 units of ADAGEN® on day 10, 14 and 18 following the bleomycin exposure. Alternatively, the mice were treated intraperitoneally with 5 units of ADAGEN® on day 10, 14 and 21. Bronchial alveolar lavage (BAL) fluid was collected from the mice to determine inflammatory cells on day 21. Mean total inflammatory cells (×10⁴)+SEM were determined using a hemocytometer. The cells were cytospun onto microscope slides and cellular differentials were performed. Data are presented as mean cells (×10⁴)+SEM, n=8 for each group.

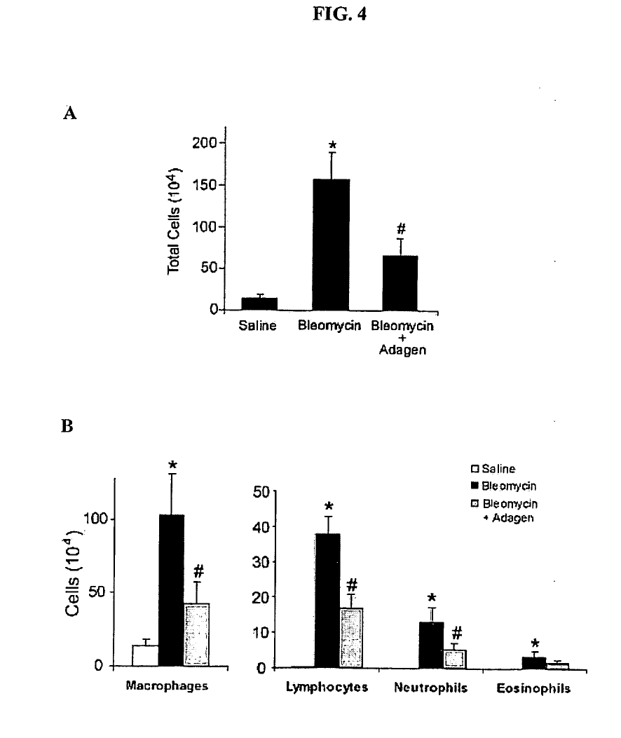

The bleomycin exposure elevated inflammatory cells in the mice which did not receive the ADAGEN® treatment. The ADAGEN® treatment significantly attenuated inflammation caused by bleomycin. The results are shown in FIG. 4A. The results also show that the ADAGEN® treatment attenuated subsets of inflammatory cells such as alveolar macrophages, lymphocytes, neutrophils and eosinophils (FIG. 4B). These cell populations were decreased by 40% relative to levels found in the lungs of bleomycin treated mice without ADAGEN® treatment. These findings as well as those of Example 2 suggest that reducing adenosine levels with ADAGEN® treatment can decrease pulmonary inflammation caused by bleomycin exposure.

### Example 5

### Effects of ADAGEN® Treatment on Collagen Production in Mice with Pulmonary Fibrosis Caused by Bleomycin

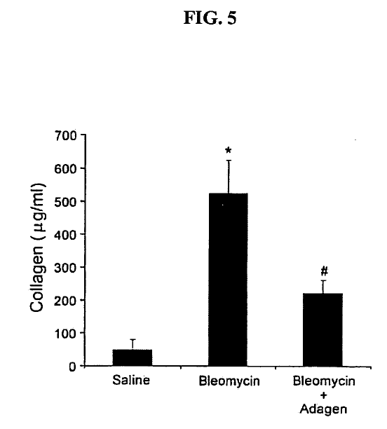

Collagen levels were examined to determine if ADAGEN® has therapeutic effects on fibrosis caused by bleomycin. BAL fluid was collected from the mice treated with ADAGEN® on day 21. The results are set forth in FIG. 5. Data are presented as mean collagen levels+SEM, n=8 for each group.

The results show that collagen levels elevated by bleomycin were significantly reduced by the ADAGEN® treatment. The mice exposed to bleomycin and treated with ADAGEN® had 45% less collagen in the airways than mice not treated with ADAGEN®. These data suggest that ADAGEN® and/or ADA have utility in the treatment of patients with pulmonary fibrosis.

We claim:

1. A method of treating an adenosine deaminase-mediated pulmonary disease, comprising administering an effective amount of adenosine deaminase to a mammal in need thereof.

2. The method of claim 1, wherein the pulmonary disease is mediated with an elevated level of adenosine.

3. The method of claim 1, wherein the pulmonary disease is selected from the group consisting of asthma, pulmonary fibrosis, cystic fibrosis and chronic obstructive pulmonary disease.

4. The method of claim 1, wherein the pulmonary disease is a chemotherapy-mediated pulmonary disease.

5. The method of claim 1 wherein the adenosine deaminase is administered by inhalation or parenterally.

6. The method of claim 1, wherein the adenosine deaminase is administered prophylactically, concurrently or subsequently with a chemotherapeutic agent.

7. The method of claim 1, wherein the adenosine deaminase is conjugated to a polyalkylene oxide.

8. The method of claim 7, wherein the polyalkylene oxide-conjugated adenosine deaminase is administered as an aerosol or as a dry powder.

9. The method of claim 1 wherein the adenosine deaminase is obtained from a bovine source.

10. The method of claim 1 wherein the adenosine deaminase is obtained from a human source.

11. The method of claim 1, wherein the adenosine deaminase is a recombinant adenosine deaminase.

12. The method of claim 7, wherein the polyalkylene oxide ranges in size from about 5,000 Daltons to about 45,000 daltons.

13. The method of claim 7, wherein the polyalkylene oxide comprises a straight, branched or multi-arm chain.

14. The method of claim 7, wherein the polyalkylene oxide is polyethylene glycol.

15. The method of claim 14, wherein the adenosine deaminase conjugated to polyethylene glycol is pegademase bovine.

16. The method of claim 1, wherein the amount of adenosine deaminase administered to the mammal is an amount sufficient to maintain plasma ADA activity in the range of from about 15 to about 35 μmol/hr/mL.

17. The method of claim 1, wherein the amount of adenosine deaminase administered to the mammal is an amount sufficient to reduce lung adenosine levels to less than about 10 nmoles per mg protein.

18. The method of claim 17, wherein the amount of adenosine deaminase administered to the mammal is an amount sufficient to reduce lung adenosine levels to less than about 5 nmoles per mg protein.

19. The method of claim 1, further comprising administering an effective dose of a second pharmacologically active agent in combination with the adenosine deaminase.

20. The method of claim 19, wherein the second pharmacologically active agent is theophylline or a bronchodilator.

21. The method of claim 19, wherein the bronchodilator is beta-adrenergic bronchodilator.

22. The method of claim 21, wherein the beta-adrenergic bronchodilator is selected from the group consisting of salmeterol, albuterol or terbutaline.

23. A kit for treating pulmonary disease in mammals, comprising adenosine deaminase and instructions for use in the treatment of pulmonary disease.

24. An inhalable formulation, comprising adenosine deaminase and a bronchodilator.

25. An inhaler suitable for use in the treatment of pulmonary conditions, comprising the inhalable formulation of claim 24 and a propellant.


SEQUENCE LISTING (8 sequences)

SEQ ID NO: 1 - protein, 356 aa, Bovine
AQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDTTLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPA

SEQ ID NO: 2 - DNA, 1074 bp, Bovine
atggctcagaccccggctttcaacaaaccgaaggtagaactgcacgtacacctggatggtgctatcaaaccggagactatcctgtactatggtcgtaagcgtggcatcgctctgccggctgacactccggaagaactgcagaacatcatcggcatggacaaaccgctgtctctgccggaattcctggctaaattcgactactacatgccggctatcgctggttgtcgtgaagcagtcaaacgtatcgcttacgaattcgtagaaatgaaagctaaagatggtgtagtatacgttgaagttcgttactctccgcacctgctggcaaactctaaagttgaaccgatcccgtggaaccaggcagaaggcgatctgactccggatgaagtagtttctctggttaaccagggtctgcaggagggtgaacgcgatttcggcgtaaaagttcgttctatcctgtgctgcatgcgccaccagccgtcttggtcttctgaagttgttgaactgtgcaagaaataccgtgagcagaccgtagttgctatcgatctggcaggtgatgaaaccatcgaaggttcttctctgtttccgggtcacgtaaaggcttatgctgaagctgttaaatctggcgtacaccgtactgtacacgcaggtgaagttggttctgctaacgttgttaaagaagctgttgacaccctgaaaactgaacgcctgggtcacggctaccacaccctggaagacaccaccctgtacaaccgtctgcgtcaggaaaacatgcacttcgaagtttgtccgtggtcctcttacctgactggtgcttggaaaccggacaccgaacacccggttgttcgtttcaaaaacgaccaggtaaactactctctgaacactgacgatccgctgatcttcaaatctaccctggacaccgactaccagatgaccaaaaacgaaatgggtttcactgaagaagaattcaaacgtctgaacatcaacgctgctaagtcctcttttctgccggaagatgagaaaaaagaactgctggacctgctgtacaaggcatacggtatgccgtctccggcttaa

SEQ ID NO: 3 - protein, 362 aa, Human
AQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 4 - DNA, 1103 bp, Human
ccatggcacaaacacccgcattcgataaacctaaagttgaactgcacgtccatttagatggcagcatcaaacctgaaaccattctgtactacggtcgccgtcgtggaatcgccctcccagcaaataccgccgaaggactgctcaacgttattggtatggacaaaccactgacactccctgatttcttagccaaatttgattattatatgcccgcaattgcaggttgccgcgaagccattaaacgcattgcttacgagtttgtggaaatgaaagctaaagaaggcgtagtttatgttgaagtccgttactctcctcatttattagcaaatagcaaagtagaacctatcccgtggaaccaagcagaaggtgatttaacacccgatgaagttgtagccttagtgggtcaaggcctccaagaaggtgaacgtgattttggtgtgaaagcacgctccatcttatgttgcatgcgccatcaaccgaactggtcccccaaagttgtagaactgtgcaaaaaataccaacaacaaaccgtagtggctattgatctggcgggtgatgaaacgatcccaggctcttccttattgccaggccacgttcaagcatatcaagaagccgttaaatctggcattcatcgtaccgttcacgctggtgaagttgggagtgcagaagtagttaaagaagcagtagatattctcaaaaccgaacgcctcggccacggctaccatactttagaagatcaagcgttatataaccgcctccgccaagaaaacatgcactttgaaatttgcccctggagttcctatttaaccggagcctggaaacctgacactgaacacgctgtgattcgcctgaaaaatgaccaagccaactattcccttaataccgacgatcccctcatttttaaatccacattagacaccgactatcaaatgaccaaacgcgacatgggttttacagaagaagaatttaaacgtctgaacatcaatgcagccaaaagctcttttcttcctgaagatgaaaaacgtgaacttttagacctgctgtataaggcttacggcatgccaccctccgcatcagcaggccaaaatctgtgataaaagctt

SEQ ID NO: 5 - protein, 356 aa, Bovine
  misc_feature (74)..(74): Cys to Ser mutein
AQTPAFNKPKVELHVHLDGAIKPETILYYGRKRGIALPADTPEELQNIIGMDKPLSLPEFLAKFDYYMPAIAGSREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDTTLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPA

SEQ ID NO: 6 - DNA, 1074 bp, Bovine
atggctcagaccccggctttcaacaaaccgaaggtagaactgcacgtacacctggatggtgctatcaaaccggagactatcctgtactatggtcgtaagcgtggcatcgctctgccggctgacactccggaagaactgcagaacatcatcggcatggacaaaccgctgtctctgccggaattcctggctaaattcgactactacatgccggctatcgctggttctcgtgaagcagtcaaacgtatcgcttacgaattcgtagaaatgaaagctaaagatggtgtagtatacgttgaagttcgttactctccgcacctgctggcaaactctaaagttgaaccgatcccgtggaaccaggcagaaggcgatctgactccggatgaagtagtttctctggttaaccagggtctgcaggagggtgaacgcgatttcggcgtaaaagttcgttctatcctgtgctgcatgcgccaccagccgtcttggtcttctgaagttgttgaactgtgcaagaaataccgtgagcagaccgtagttgctatcgatctggcaggtgatgaaaccatcgaaggttcttctctgtttccgggtcacgtaaaggcttatgctgaagctgttaaatctggcgtacaccgtactgtacacgcaggtgaagttggttctgctaacgttgttaaagaagctgttgacaccctgaaaactgaacgcctgggtcacggctaccacaccctggaagacaccaccctgtacaaccgtctgcgtcaggaaaacatgcacttcgaagtttgtccgtggtcctcttacctgactggtgcttggaaaccggacaccgaacacccggttgttcgtttcaaaaacgaccaggtaaactactctctgaacactgacgatccgctgatcttcaaatctaccctggacaccgactaccagatgaccaaaaacgaaatgggtttcactgaagaagaattcaaacgtctgaacatcaacgctgctaagtcctcttttctgccggaagatgagaaaaaagaactgctggacctgctgtacaaggcatacggtatgccgtctccggcttaa

SEQ ID NO: 7 - protein, 362 aa, Human
  misc_feature (74)..(74): Cys to Ser mutein
AQTPAFDKPKVELHVHLDGSIKPETILYYGRRRGIALPANTAEGLLNVIGMDKPLTLPDFLAKFDYYMPAIAGSREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 8 - DNA, 1089 bp, Human
atggctcagacacccgcatttgataaaccgaaagtggaactgcatgtccacctggatggtagcatcaaaccggaaactatcttatattacggtcggcgtcgtggtattgcgttgccggcaaacacggctgaaggtttgctgaacgtgatcggcatggacaaaccgctgaccttgccggattttttggcgaaatttgattattatatgccggcgattgctggttcccgcgaggcaatcaaacgcatcgcgtatgagtttgttgaaatgaaagcgaaagaaggcgttgtgtatgttgaggtccgttacagtccgcatctgctggctaacagcaaggtagaacctatcccctggaaccaagctgaaggcgatctgacgccggatgaagtggttgctctggtgggtcagggtttacaggagggggagcgcgattttggcgttaaagctcgctctattttatgttgcatgcgccatcagcccaattggtccccgaaagtggttgaactttgtaaaaagtaccaacaacagaccgttgtcgcgattgatttggcaggcgatgaaacaattccaggcagctccctgttgccagggcacgtgcaagcgtaccaagaagcagtgaaaagcggcatccaccggactgtccacgccggcgaggtcggtagcgccgaggttgtgaaagaagccgtggacatcctgaaaaccgagcggctgggccatgggtaccacacactggaggatcaggcattatataaccgcttacgccaggaaaatatgcatttcgaaatttgtccgtggagtagttacttaactggcgcgtggaaaccggataccgaacatgcggttatccgcttaaagaatgatcaagcaaattacagtctgaatacagatgatcccctgattttcaagtctaccctggacacagattatcagatgacgaagcgggatatgggatttacggaagaagaatttaagcgtctcaatatcaatgcggcgaaatcttcatttctgccggaagatgagaaacgtgagttgctggatcttctgtacaaggcctacggtatgccgccgagcgcatcggccgggcagaacctg